(19) United States
(12) Patent Application Publication
(10) Pub. No.: US 2016/0242970 A1
(43) Pub. Date: Aug. 25, 2016

(54) ONE-WAY VALVE NONWOVEN MATERIAL

(71) Applicants: KIMBERLY-CLARK WORLDWIDE, INC., Neenah, WI (US); THE BOARD OF TRUSTEES OF THE UNIVERSITY OF ILLINOI, Urbana, IL (US)

(72) Inventors: Jian Qin, Appleton, WI (US); Donald E. Waldroup, Roswell, GA (US); Constantine Michael Megaridis, Oak Park, IL (US); Joseph Edward Mates, Highland, IN (US); Thomas Michael Schutzius, Tinley Park, IL (US)

(21) Appl. No.: 15/033,343

(22) PCT Filed: Oct. 30, 2014
(86) PCT No.: PCT/US14/63036

§ 371 Date: Apr. 29, 2016

Related U.S. Application Data
(60) Provisional application No. 61/898,126, filed on Oct. 31, 2013.
(60) Provisional application No. 61/908,506, filed on Nov. 25, 2013.

(51) Int. Cl. A61F 13/511; A61L 15/26; A61L 15/52; A61L 15/18

(52) U.S. Cl. CPC A61F 13/51121; A61L 15/18; A61L 15/26; A61L 15/52; A61F 2013/5103

(57) ABSTRACT

A material having one-way valve properties includes a nonwoven substrate having a first surface having a first surface hydrohead value and a second surface having a second surface hydrohead value; and a superhydrophobic formulation disposed on the first surface, wherein the first surface hydrohead value is less than about 1 cm, and wherein the second surface hydrohead value is at least 4 cm greater than the first surface hydrohead value. Also, a personal care article includes this nonwoven substrate in a nonwoven fluid permeable topsheet having a body-facing surface and an opposing backside surface, the article also including a fluid impermeable backsheet and at least one intermediate layer disposed therebetween.

### PRIORITY INFORMATION

The present application claims priority to U.S. Provisional Patent Application Ser. No. 61/898,126 titled “One-Way Valve Nonwoven Material” filed on Oct. 31, 2013 and to U.S. Provisional Patent Application Ser. No. 61/908,506 titled “One-Way Valve Nonwoven Material” filed on Nov. 25, 2013, the disclosures of which are incorporated by reference herein.

### BACKGROUND

The present disclosure relates to nonwoven materials for personal care products, particularly disposable absorbent articles, that aid in keeping the surface of the article appearing and feeling clean.

A number of disposable, personal care articles that collect body fluids exist; however, their tendency to leak off the surface before the liquid absorbent capacity is entirely used is an ongoing challenge that faces many manufacturers. Additionally, certain fluids, such as menses and runny BM (feces), have viscoelastic properties that make obtaining good intake and distribution performance particularly problematic. In particular, the relatively high viscosity and/or elasticity of such fluids tend to interfere with the absorption and distribution of the fluids within the absorbent article. In other instances, intake performance of an absorbent article can be impeded when components of the menses block the channels between the particles or fibers contained in the absorbent article. This phenomenon is often referred to as fouling. Although attempts have been made to improve the effects of fouling through modification of the viscoelastic properties of the fluid itself, actual improvement into the absorbent article still needs development.

In addition to problems with leakage in some disposable, personal care articles, there are also hygienic issues that directly affect the user. Often the body fluid sits in direct contact with the user that makes for an unpleasant and unclean feel. Particularly with feminine hygiene products such as sanitary napkins, the unpleasant or unclean feeling that can often be caused by bodyside liner stains can lead to poor perception in product performance and the inability to get maximum use from the product.

### SUMMARY

Therefore, there is a need in the art for a treatment that can be used in connection with personal care products, such as absorbent articles, that provides improved intake and distribution performance, reduced leakage, reduced stains, reduced surface rewet or flowback for an overall cleaner, drier, and more pleasant feel and user experience.

The present disclosure provides a material having one-way valve properties, the material including a nonwoven substrate having a first surface having a first surface hydrohead value and a second surface having a second surface hydrohead value; and a superhydrophobic formulation disposed on the first surface, wherein the first surface hydrohead value is less than about 1 cm, and wherein the second surface hydrohead value is at least 4 cm greater than the first surface hydrohead value.

The present disclosure also provides a material having one-way valve properties, the material including a nonwoven substrate having a first surface having a first surface hydrohead value and a second surface having a second surface hydrohead value; and a superhydrophobic formulation disposed on the first surface at an add-on level of less than about 2 gsm, wherein the second surface hydrohead value is at least 4 cm greater than the first surface hydrohead value.

The present disclosure also provides a personal care article including a nonwoven fluid permeable topsheet having a body-facing surface and an opposing backside surface, a fluid impermeable backsheet, and at least one intermediate layer disposed therebetween, wherein fluid permeable topsheet includes a nonwoven substrate having a first surface having a first surface hydrohead value and a second surface having a second surface hydrohead value; and a superhydrophobic formulation disposed on the first surface, wherein the first surface hydrohead value is less than about 1 cm, and wherein the second surface hydrohead value is at least 4 cm greater than the first surface hydrohead value.

### BRIEF DESCRIPTION OF THE DRAWINGS

The foregoing and other features and aspects of the present disclosure and the manner of attaining them will become more apparent, and the disclosure itself will be better understood by reference to the following description, appended claims and accompanying drawings, where:

FIG. 1 illustrates a non-wettable porous substrate resisting penetration of water due to its small pore size d and high hydrophobicity (high contact angle, θ);

FIG. 2A schematically illustrates the working mechanism of the one-way valve of the present disclosure with a coating down;

FIG. 2B schematically illustrates the working mechanism of the one-way valve of the present disclosure with a coating up;

FIG. 3 illustrates a scanning electron microscope (SEM) image of a cross section of a standard paper towel treated as described herein, where the left inset displays the surface morphology of a hydrophobic coated fiber, compared with that of an uncoated hydrophilic fiber displayed in the right inset;

FIG. 4A illustrates the phenomenon that coated-on-one-side substrate HDPT introduced to pressures with the coating side up passes fluid more easily than with the coating side down, creating a valve window;

FIG. 4B illustrates the phenomenon that coated-on-one-side substrate SPT introduced to pressures with the coating side up passes fluid more easily than with the coating side down, creating a valve window; and

FIG. 5 illustrates the phenomenon of oil-water separation for coated-on-one-side substrates introduced to an oil-water mixture, or emulsion.

Repeat use of reference characters in the present specification and drawings is intended to represent the same or analogous features or elements of the present disclosure. The drawings are representational and are not necessarily drawn to scale. Certain proportions thereof might be exaggerated, while others might be minimized.

### DETAILED DESCRIPTION

While the specification concludes with the claims particularly pointing out and distinctly claiming the disclosure, it is believed that the present disclosure will be better understood from the following description.

All percentages, parts and ratios are based upon the total weight of the compositions of the present disclosure, unless otherwise specified. All such weights as they pertain to listed ingredients are based on the active level and, therefore, do not include solvents or by-products that can be included in commercially available materials, unless otherwise specified. The term “weight percent” can be denoted as “wt. %” herein. Except where specific examples of actual measured values are presented, numerical values referred to herein should be considered to be qualified by the word “about”.

As used herein, “comprising” means that other steps and other ingredients that do not affect the end result can be added. This term encompasses the terms “consisting of” and “consisting essentially of.” The compositions and methods/processes of the present disclosure can comprise, consist of, and consist essentially of the essential elements and limitations of the disclosure described herein, as well as any of the additional or optional ingredients, components, steps, or limitations described herein.

As used herein, the phrase “absorbent article” generally refers to devices that absorb and contain body fluids, and more specifically, refers to devices that are placed against or near the skin to absorb and contain the various fluids discharged from the body and, in particular, viscoelastic fluids. Examples of absorbent articles include, but are not limited to, absorbent articles intended for personal wear, such as diapers; incontinence products; feminine hygiene products, such as feminine napkins, panty liners, tampons, and interlabial pads; other personal garments; and the like.

As used herein, “fouling” means the change in permeability of a fluid as it passes through a porous medium. More particularly, fouling is the reduction in permeability that occurs when components of a fluid pass through a porous medium and interact with the material structure, decreasing the inherent permeability of the porous material.

The term “hydrophilic,” as used herein, refers to surfaces with water contact angles well below 90°.

The term “hydrophobic,” as used herein, refers to the property of a surface to repel water with a water contact angle from about 90° to about 120°.

As used herein, “rewetting” refers to the amount of fluid that comes from the absorbent core back into and through the top layer, nonwoven surface. This can also be referred to as “flowback.”

The term “superhydrophobic” refers to the property of a surface to repel water very effectively. This property is quantified by a water contact angle generally exceeding 150°.

The present disclosure relates to improved personal care products, particularly disposable absorbent articles. Personal care products of the present disclosure include, but are not limited to, feminine hygiene products like sanitary wipes and menses absorbing devices (e.g., sanitary napkins and tampons), infant and child care products such as disposable diapers, absorbent underpants, and training pants, wound dressings such as bandages, incontinent products, products for wiping and absorbing oils and/or organic solvents, and the like.

Disposable absorbent articles such as the feminine care absorbent product, for example, can include a liquid permeable topsheet, a substantially liquid impermeable backsheet joined to the topsheet, and an absorbent core positioned and held between the topsheet and the backsheet. The topsheet is operatively permeable to the liquids that are intended to be held or stored by the absorbent article, and the backsheet can be substantially impermeable or otherwise operatively impermeable to the intended liquids. The absorbent article can also include an additional layer(s). This additional layer(s) can be a liquid intake layer, liquid wicking layers, liquid distribution layers, transfer layers, barrier layers, and the like, as well as combinations thereof. Disposable absorbent articles and the components thereof can operate to provide a body-facing surface (top surface of the topsheet) and a garment-facing surface (back surface of the backsheet). As used herein, the “body-facing” or “bodyside” surface refers to the surface of the topsheet that is disposed toward or placed adjacent to the body of the wearer during ordinary use. The “garment-side surface” refers to the backsheet where the back of the surface is disposed away from the wearer's body and adjacent to the garment of the wearer during ordinary use. Suitable absorbent articles are described in more detail in U.S. Pat. No. 7,632,258.

The fluid permeable topsheet of the present disclosure can be left untreated or can be treated with a superhydrophobic composition that helps to keep fluids from sitting atop the surface that can leave an unpleasant and/or unclean feeling from stains, accumulated debris, or wetness on the surface. The disposable absorbent articles of the present disclosure are particularly adapted to receive fluids having viscoelastic properties, such as menses, mucous, blood products, and feces, among others to reduce stain area, reduce rewet, improve fluid intake, distribution, absorption properties, and decrease leakage.

Although the present disclosure is discussed primarily in combination with feminine hygiene products such as feminine napkins, panty liners, and interlabial pads, it will be readily apparent to one skilled in the art based on the disclosure that the products and methods described herein can also be used in combination with numerous other absorbent articles designed to absorb fluids other than menses such as runny BM, urine, and the like.

In addition, the superhydrophobic nature of the coating and its relative orientation also allows for separation of fluids of differing surface energies such as oil and water, as described below.

The absorbent articles of the present disclosure include a fluid permeable topsheet that is preferably a nonwoven, body-facing fibrous sheet material. The present disclosure provides an advantage over topsheets including a thermoplastic film because nonwovens are generally softer, cause less sweating and irritation from sweat, and avoid the plastic feel or rustling that is often associated with plastics and films.

Nonwovens of the present disclosure include, but are not limited to, paper toweling, spunbond, meltblown, coform, air-laid, bonded-carded web materials, hydroentangled (spunlace) materials, combinations thereof, and the like. For example, the fibers from which the nonwoven material is made can be produced by the meltblowing or spunbonding processes, including those producing bicomponent, biconstituent or polymer blend fibers that are well known in the art. These processes generally use an extruder to supply melted thermoplastic polymer to a spinneret where the polymer is fiberized to yield fibers that can be staple length or longer. The fibers are then drawn, usually pneumatically, and deposited on a moving formations mat or belt to form the nonwoven fabric. The fibers produced in the spunbond and meltblown processes can be microfibers. Microfibers of the present disclosure are small diameter fibers having an average diameter not greater than about 75 microns, for example, having an average diameter of from about 0.5 microns to about 50 microns, or more particularly, microfibers can have an average diameter of from about 2 microns to about 40 microns.

Suitable substrates of the present disclosure can include a nonwoven fabric, woven fabric, knit fabric, or laminates of these materials. The substrate can also be a tissue or towel, as described herein. Materials and processes suitable for forming such substrate are generally well known to those skilled in the art. For instance, some examples of nonwoven fabrics that can be used in the present disclosure include, but are not limited to, paper toweling, spunbonded webs, meltblown webs, bonded carded webs, air-laid webs, coform webs, spunlace or hydraulically entangled webs, and the like. In each case, at least one of the fibers used to prepare the nonwoven fabric is a thermoplastic material containing fiber. In addition, nonwoven fabrics can be a combination of thermoplastic fibers and natural fibers, such as, for example, cellulosic fibers (softwood pulp, hardwood pulp, thermomechanical pulp, etc.). Generally, from the standpoint of cost and desired properties, the substrate of the present disclosure is a nonwoven fabric.

If desired, the nonwoven fabric can also be bonded using techniques well known in the art to improve the durability, strength, hand, aesthetics, texture, and/or other properties of the fabric. For instance, the nonwoven fabric can be thermally (e.g., pattern bonded, through-air dried), ultrasonically, adhesively and/or mechanically (e.g. needled) bonded. For instance, various pattern bonding techniques are described in U.S. Pat. No. 3,855,046 to Hansen; U.S. Pat. No. 5,620,779 to Levy, et al.; U.S. Pat. No. 5,962,112 to Haynes, et al.; U.S. Pat. No. 6,093,665 to Sayovitz, et al.; U.S. Design Pat. No. 428,267 to Romano, et al.; and U.S. Design Pat. No. 390,708 to Brown.

The nonwoven fabric can be bonded by continuous seams or patterns. As additional examples, the nonwoven fabric can be bonded along the periphery of the sheet or simply across the width or cross-direction of the web adjacent the edges. Other bond techniques, such as a combination of thermal bonding and latex impregnation, can also be used. Alternatively and/or additionally, a resin, latex or adhesive can be applied to the nonwoven fabric by, for example, spraying or printing, and dried to provide the desired bonding. Still other suitable bonding techniques can be described in U.S. Pat. No. 5,284,703 to Everhart, et al., U.S. Pat. No. 6,103,061 to Anderson, et al., and U.S. Pat. No. 6,197,404 to Varona.

In another aspect, the substrate of the present disclosure is formed from a spunbonded web containing monocomponent and/or multicomponent fibers. Multicomponent fibers are fibers that have been formed from at least two polymer components. Such fibers are usually extruded from separate extruders but spun together to form one fiber. The polymers of the respective components are usually different from each other although multicomponent fibers can include separate components of similar or identical polymeric materials. The individual components are typically arranged in substantially constantly positioned distinct zones across the cross-section of the fiber and extend substantially along the entire length of the fiber. The configuration of such fibers can be, for example, a side-by-side arrangement, a pie arrangement, or any other arrangement.

When used, multicomponent fibers can also be splittable. In fabricating multicomponent fibers that are splittable, the individual segments that collectively form the unitary multicomponent fiber are contiguous along the longitudinal direction of the multicomponent fiber in a manner such that one or more segments form part of the outer surface of the unitary multicomponent fiber. In other words, one or more segments are exposed along the outer perimeter of the multicomponent fiber. For example, splittable multicomponent fibers and methods for making such fibers are described in U.S. Pat. No. 5,935,883 to Pike and U.S. Pat. No. 6,200,669 to Marmon, et al.

The substrate of the present disclosure can also contain a coform material. The term “coform material” generally refers to composite materials including a mixture or stabilized matrix of thermoplastic fibers and a second non-thermoplastic material. As an example, coform materials can be made by a process in which at least one meltblown die head is arranged near a chute through which other materials are added to the web while it is forming. Such other materials can include, but are not limited to, fibrous organic materials such as woody or non-woody pulp such as cotton, rayon, recycled paper, pulp fluff and also superabsorbent particles, inorganic absorbent materials, treated polymeric staple fibers and the like. Some examples of such coform materials are disclosed in U.S. Pat. No. 4,100,324 to Anderson, et al.; U.S. Pat. No. 5,284,703 to Everhart, et al.; and U.S. Pat. No. 5,350,624 to Georger, et al.

Additionally, the substrate can also be formed from a material that is imparted with texture one or more surfaces. For instances, in some aspects, the substrate can be formed from a dual-textured spunbond or meltblown material, such as described in U.S. Pat. No. 4,659,609 to Lamers, et al. and U.S. Pat. No. 4,833,003 to Win, et al.

In one particular aspect of the present disclosure, the substrate is formed from a hydroentangled nonwoven fabric. Hydroentangling processes and hydroentangled composite webs containing various combinations of different fibers are known in the art. A typical hydroentangling process utilizes high pressure jet streams of water to entangle fibers and/or filaments to form a highly entangled consolidated fibrous structure, e.g., a nonwoven fabric. Hydroentangled nonwoven fabrics of staple length fibers and continuous filaments are disclosed, for example, in U.S. Pat. No. 3,494,821 to Evans and U.S. Pat. No. 4,144,370. Hydroentangled composite nonwoven fabrics of a continuous filament nonwoven web and a pulp layer are disclosed, for example, in U.S. Pat. No. 5,284,703 to Everhart, et al. and U.S. Pat. No. 6,315,864 to Anderson, et al.

Of these nonwoven fabrics, hydroentangled nonwoven webs with staple fibers entangled with thermoplastic fibers is especially suited as the substrate. In one particular example of a hydroentangled nonwoven web, the staple fibers are hydraulically entangled with substantially continuous thermoplastic fibers. The staple can be cellulosic staple fiber, non-cellulosic stable fibers or a mixture thereof. Suitable non-cellulosic staple fibers includes thermoplastic staple fibers, such as polyolefin staple fibers, polyester staple fibers, nylon staple fibers, polyvinyl acetate staple fibers, and the like or mixtures thereof. Suitable cellulosic staple fibers include for example, pulp, thermomechanical pulp, synthetic cellulosic fibers, modified cellulosic fibers, and the like. Cellulosic fibers can be obtained from secondary or recycled sources. Some examples of suitable cellulosic fiber sources include virgin wood fibers, such as thermomechanical, bleached and unbleached softwood and hardwood pulps. Secondary or recycled cellulosic fibers can be obtained from office waste, newsprint, brown paper stock, paperboard scrap, etc., can also be used. Further, vegetable fibers, such as abaca, flax, milkweed, cotton, modified cotton, cotton linters, can also be used as the cellulosic fibers. In addition, synthetic cellulosic fibers such as, for example, rayon and viscose rayon can be used. Modified cellulosic fibers are generally are composed of derivatives of cellulose formed by substitution of appropriate radicals (e.g., carboxyl, alkyl, acetate, nitrate, etc.) for hydroxyl groups along the carbon chain.

One particularly suitable hydroentangled nonwoven web is a nonwoven web composite of polypropylene spunbond fibers, which are substantially continuous fibers, having pulp fibers hydraulically entangled with the spunbond fibers. Another particularly suitable hydroentangled nonwoven web is a nonwoven web composite of polypropylene spunbond fibers having a mixture of cellulosic and non-cellulosic staple fibers hydraulically entangled with the spunbond fibers.

The substrate of the present disclosure can be prepared solely from thermoplastic fibers or can contain both thermoplastic fibers and non-thermoplastic fibers. Generally, when the substrate contains both thermoplastic fibers and non-thermoplastic fibers, the thermoplastic fibers make up from about 10% to about 90%, by weight of the substrate. In a particular aspect, the substrate contains between about 10% and about 30%, by weight, thermoplastic fibers.

Generally, a nonwoven substrate will have a basis weight in the range of about 17 gsm (grams per square meter) to about 200 gsm, more typically, between about 33 gsm to about 200 gsm. The actual basis weight can be higher than 200 gsm, but for many applications, the basis weight will be in the 33 gsm to 150 gsm range.

The thermoplastic materials or fibers making-up at least a portion of the substrate can essentially be any thermoplastic polymer. Suitable thermoplastic polymers include polyolefins, polyesters, polyamides, polyurethanes, polyvinylchloride, polytetrafluoroethylene, polystyrene, polyethylene terephthalate, biodegradable polymers such as polylactic acid, and copolymers and blends thereof. Suitable polyolefins include polyethylene, e.g., high density polyethylene, medium density polyethylene, low density polyethylene and linear low density polyethylene; polypropylene, e.g., isotactic polypropylene, syndiotactic polypropylene, blends of isotactic polypropylene and atactic polypropylene, and blends thereof; polybutylene, e.g., poly(1-butene) and poly(2-butene); polypentene, e.g., poly(1-pentene) and poly(2-pentene); poly(3-methyl-1-pentene); poly(4-methyl 1-pentene); and copolymers and blends thereof. Suitable copolymers include random and block copolymers prepared from two or more different unsaturated olefin monomers, such as ethylene/propylene and ethylene/butylene copolymers. Suitable polyamides include nylon 6, nylon 6/6, nylon 4/6, nylon 11, nylon 12, nylon 6/10, nylon 6/12, nylon 12/12, copolymers of caprolactam and alkylene oxide diamine, and the like, as well as blends and copolymers thereof. Suitable polyesters include polyethylene terephthalate, polytrimethylene terephthalate, polybutylene terephthalate, polytetramethylene terephthalate, polycyclohexylene-1,4-dimethylene terephthalate, and isophthalate copolymers thereof, as well as blends thereof. These thermoplastic polymers can be used to prepare both substantially continuous fibers and staple fibers, in accordance with the present disclosure.

In another aspect, the substrate can be a tissue product. The tissue product can be of a homogenous or multi-layered construction, and tissue products made therefrom can be of a single-ply or multi-ply construction. The tissue product desirably has a basis weight of about 10 g/m2 to about 65 g/m2, and density of about 0.6 g/cc or less. More desirably, the basis weight will be about 40 g/m2 or less and the density will be about 0.3 g/cc or less. Most desirably, the density will be about 0.04 g/cc to about 0.2 g/cc. Unless otherwise specified, all amounts and weights relative to the paper are on a dry basis. Tensile strengths in the machine direction can be in the range of from about 100 to about 5,000 grams per inch of width. Tensile strengths in the cross-machine direction are from about 50 grams to about 2,500 grams per inch of width. Absorbency is typically from about 5 grams of water per gram of fiber to about 9 grams of water per gram of fiber.

Conventionally pressed tissue products and methods for making such products are well known in the art. Tissue products are typically made by depositing a papermaking furnish on a foraminous forming wire. Once the furnish is deposited on the forming wire, it is referred to as a web. The web is dewatered by pressing the web and drying at elevated temperature. The particular techniques and typical equipment for making webs according to the process just described are well known to those skilled in the art. In a typical process, a low consistency pulp furnish is provided from a pressurized headbox, which has an opening for delivering a thin deposit of pulp furnish onto the forming wire to form a wet web. The web is then typically dewatered to a fiber consistency of from about 7% to about 25% (total web weight basis) by vacuum dewatering and further dried by pressing operations wherein the web is subjected to pressure developed by opposing mechanical members, for example, cylindrical rolls. The dewatered web is then further pressed and dried by a steam drum apparatus known in the art as a Yankee dryer. Pressure can be developed at the Yankee dryer by mechanical means such as an opposing cylindrical drum pressing against the web. Multiple Yankee dryer drums can be employed, whereby additional pressing is optionally incurred between the drums. The formed sheets are considered to be compacted since the entire web is subjected to substantial mechanical compressional forces while the fibers are moist and are then dried while in a compressed state. In other aspects, the tissue can be formed by creping as is known in the art.

One particular aspect of the present disclosure utilizes an uncreped through-air-drying technique to form the tissue product. Through-air-drying can increase the bulk and softness of the web. Examples of such a technique are disclosed in U.S. Pat. No. 5,048,589 to Cook, et al.; U.S. Pat. No. 5,399,412 to Sudall, et al.; U.S. Pat. No. 5, 510,001 to Hermans, et al.; U.S. Pat. No. 5,591,309 to Ruqowski, et al.; U.S. Pat. No. 6,017,417 to Wendt, et al., and U.S. Pat. No. 6,432,270 to Liu, et al. Uncreped through-air-drying generally involves the steps of: (1) forming a furnish of cellulosic fibers, water, and optionally, other additives; (2) depositing the furnish on a traveling foraminous belt, thereby forming a fibrous web on top of the traveling foraminous belt; (3) subjecting the fibrous web to through-air-drying to remove the water from the fibrous web; and (4) removing the dried fibrous web from the traveling foraminous belt.

The nonwoven of the present disclosure can also be a multilayer laminate. An example of a multilayer laminate is an aspect wherein some of the layers are spunbond and some meltblown such as a spunbond/meltblown/spunbond (SMS) laminate as disclosed in U.S. Pat. No. 4,041,203 to Brock et al., U.S. Pat. No. 5,169,706 to Collier, et al., and U.S. Pat. No. 4,374,888 to Bornslaeger. Such a laminate can be made by sequentially depositing onto a moving forming belt first a spunbond fabric layer, then a meltblown fabric layer and last another spunbond layer and then bonding the laminate in a manner described below. Alternatively, the fabric layers can be made individually, collected in rolls, and combined in a separate bonding step. Such fabrics usually have a basis weight of from about 0.1 to 12 OSY (ounces per square yard) (6 to 400 gsm), or more particularly from about 0.75 to about 3 OSY.

The present disclosure uses superhydrophobic compositions to treat a nonwoven topsheet to significantly reduce re-wetting and staining that often occurs during the use of personal care products such as disposable absorbent articles. The treated topsheet functions as a “one-way valve” that allows fluid to more easily migrate from the body-facing surface to an absorbent core. Due to the design and composition used in the present disclosure, there is less undesirable re-wetting or flowback caused by the fluid re-entering from the absorbent core to the body-facing surface of the topsheet. One-way valve performance means that liquid passes through in one direction, but does not pass through in the opposite direction under standard conditions. This characteristic is a form of diodicity in that the topsheet acts like a diode in electricity.

One way to achieve a “one-way valve” in the nonwoven topsheet of the present disclosure is with strategic superhydrophobic treatments. Use of a “one way valve” system drives the fluid into the absorbent layer, away from the consumer's body, such that the consumer feels more dry and clean for an overall improved performance.

To achieve the one way valve behavior, only one of the two surfaces of a nonwoven substrate is to be treated with a superhydrophobic coating at a level high enough to enable the surface superhydrophobicity, but low enough to keep the nonwoven substrate's pore structure open so that water can penetrate through the pores.

Experimentation has demonstrated that the preferred treatment of the nonwoven substrate is to have one surface coated with a superhydrophobic formulation at an add-on level of about less than 2 gsm. The nonwoven substrate thereby exhibits at least about 16 cm water height difference in hydrohead values when measured from the two different sides of the nonwoven substrate.

To achieve a height difference in hydrohead values of about 4 cm water when measured at the two different surfaces of the nonwoven substrate, the add-on level of a superhydrophobic formulation is critical. If the add-on level is too low, the two surfaces are quite similar in terms of their hydrophilic/hydrophobic properties because the two surfaces are two sides of the same material. Because the hydrohead values of the two surfaces in this instance are quite similar, the difference between them is quite small, near or equal to 0 cm.

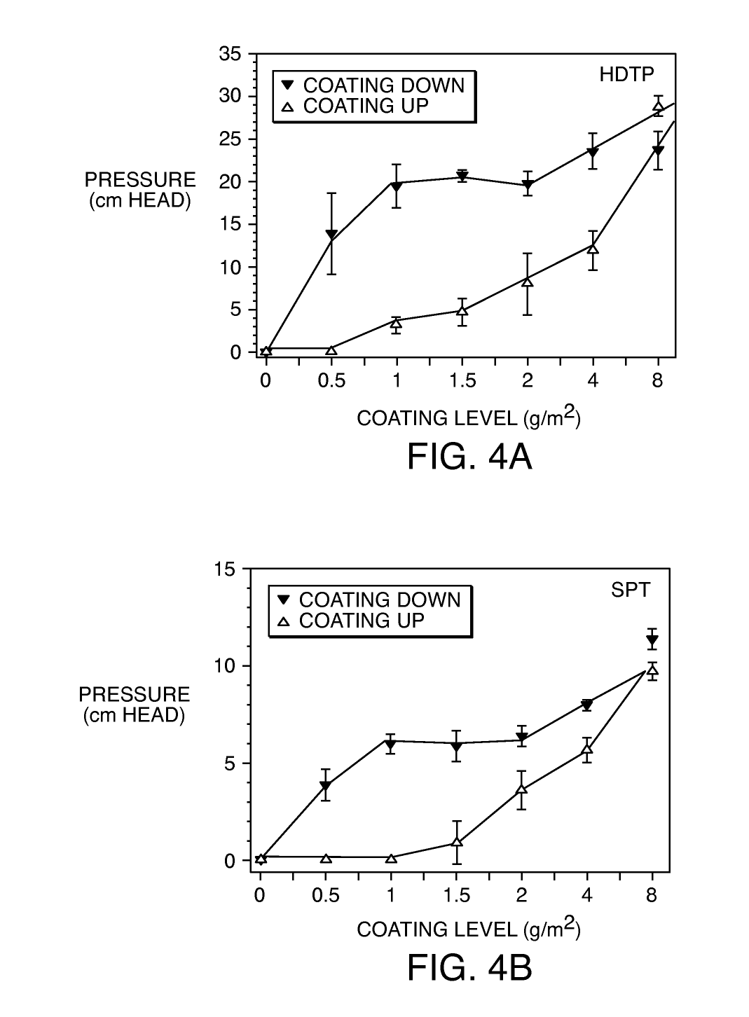

Conversely, if the add-on level is too high (>>2 gsm), the superhydrophobic formulation forms a layer of continuous film on the treated side. This film layer completely blocks liquid from penetrating from both sides. This results in a very high hydrohead value for each surface. Again, because the hydrohead values of the two surfaces in this instance are quite similar, the difference between them is also quite small, near or equal to 0 cm. Therefore, the desired add-on level between the minimum and maximum add-on level is the add-on level range in which the one-way valve behavior can be achieved. FIGS. 4A and 4B illustrate this trend.

The add-on level range is also dependent upon the structural parameters of a substrate, such as fiber size, porosity, density, uniformity, and basis weight. When a substrate has relatively larger diameter fibers, higher porosity, lower density, less uniform and/or lower basis weight, the desired add-on range in FIGS. 4A and 4B will shift toward the right, meaning both the minimum and maximum add-on levels will have higher values. On the other hand, when a substrate has relatively smaller diameter fibers, lower porosity, higher density, more uniform and/or higher basis weight, the add-on range in FIGS. 4A and 4B will shift toward the left, meaning both the minimum and maximum add-on levels will have lower values.

Conventional scalable methods, such as spraying, can be used to apply a superhydrophobic coating on a surface. In one aspect, a hydrophilic nano-structured filler (NANOMER PGV nanoclay from Sigma Aldrich) such as a bentonite clay without organic modification is used. As a hydrophobic component, a 20 wt. % dispersion of a fluorinated ethylene-acrylic co-polymer (PMC) in water is used, as obtained from DuPont (trade name is CAPSTONE ST-100). The hydrophilic nanoclay is added to water and is sonicated until a stable suspension is produced. Sonication can be done by using a probe sonicator at room temperature (SONICS 750 W, High Intensity Ultrasonic Processor, 13 mm diameter tip at 30% amplitude). At these settings, it can take from about 15 to about 30 min for a stable 15.5 g nanoclay-water suspension to form. The concentration of the nanoclay in water is kept below 2 wt. % of total suspension to prevent the formation of a gel, which renders the dispersion too viscous to spray. After placing the stable clay-water suspension under mechanical mixing at room temperature, the aqueous PMC dispersion is added drop-wise to the suspension to produce the final dispersion for spray. In such aspect, the concentrations of each component in the final dispersion for producing a superhydrophobic coating will be as follows: 95.5 wt. % water, 2.8% PMC, 1.7% nanoclay; or 97.5 wt. % water, 1.25% PMC, 1.25% nanoclay. Coatings can be applied by spray onto cellulosic substrates at a distance of about 15 to about 25 cm using an airbrush atomizer (Paasche VL siphon feed, 0.55 mm spray nozzle) either by hand or by mounting the device onto an industrial fluid dispensing robot (EFD, Ultra TT Series). EFD nozzles with air assist can also be used as this achieves extremely fine mists during spray application. The smallest nozzle diameter suggested for the EFD dispensing system is about 0.35 mm. The air fans assist in shaping the spray cone into an oval shape, which is useful for producing a continuous uniform coating on a linearly moving substrate. For the airbrush, operation relies on pressurized air passing through the nozzle to siphon-feed the particle dispersion and also to facilitate fluid atomization at the nozzle exit. The pressure drop applied across the sprayer can vary from about 2.1 to about 3.4 bar, depending on conditions.

Some technical difficulties are typically encountered when spraying water-based dispersions. The first major problem is insufficient evaporation of the fluid during atomization and a high degree of wetting of the dispersion onto the coated substrate, both resulting in non-uniform coatings due to contact line pinning and the so called “coffee-stain effect” when the water eventually evaporates. The second major challenge is the relatively large surface tension of water when compared with other solvents used for spray coating. Water, due to its high surface tension, tends to form non-uniform films in spray applications, thus requiring great care to ensure that a uniform coating is attained. This is especially critical for hydrophobic substrates where the water tends to bead and roll. It was observed that the best approach for applying the aqueous dispersions of the present disclosure was to produce extremely fine droplets during atomization, and to apply only very thin coatings, so as not to saturate the substrate and re-orient hydrogen bonding within the substrate that, after drying, would cause cellulosic substrates (e.g. paper towel) to become stiff.

In another aspect, the coatings are spray cast first on a substrate, such as standard paperboard or other cellulosic substrate; multiple spray passes are used to achieve different coating thicknesses. The sprayed films are then subjected to drying in an oven at about 80° C. for about 30 min to remove all excess water. The size of the substrate can be approximately, but not limited to, about 7.5 cm×9 cm. Once dried, the coatings are characterized for wettability (i.e., hydrophobic vs. hydrophilic). The substrates can be weighed on a microbalance (Sartorius® LE26P) before and after coating and drying in order to determine the minimum level of coating required to induce superhydrophobicity. This “minimum coating” does not strictly mean that the sample will resist penetration by liquids, but rather that a water droplet will bead on the surface and roll off unimpeded. Liquid repellency of substrates before and after coating can be characterized by a hydrostatic pressure setup that determines liquid penetration pressures (in cm of liquid).

Contact angle values can be obtained by a backlit optical image setup utilizing a CCD camera. For dynamic contact angle hysteresis measurements (that designate the self-cleaning property), the CCD camera can be replaced by a high-speed camera, such as a REDLAKE Motion Pro camera, to accurately capture advancing and receding contact angle values. The lower the difference between advancing and receding contact angles (i.e. contact angle hysteresis), the more self-cleaning the surface is. Liquid penetration pressure can be determined by increasing the hydrostatic column pressure until liquid penetrates the sample in accordance with ASTM F903-10. Liquid penetration can be recorded by an optical image setup utilizing a CCD camera.

Wettability of the composite coatings can be first tested on paperboard, an untextured hydrophilic cellulosic substrate deemed to be representative of the general class of cellulosic substrates (textured or untextured). Nanoclay concentration is incorporated at increasing concentrations in the coating until self-cleaning behavior is observed. The purpose of adding nanoclay to the composite coating is to affect the texture of the coating. It is known that superhydrophobicity and self-cleaning behavior are controlled by two mechanisms, namely, surface roughness and surface energy. It has also been shown that hierarchical structures in conjunction with low-surface energy groups offer an excellent pathway for achieving the roughness necessary for superhydrophobicity. Nanoclay has a platelet structure with nanoscale thickness and microscale length that, when self-assembled (through electrostatic interaction), produces the aforementioned hierarchical structure. The level of nanoclay concentration in the composite coating where self-cleaning is first observed is about 38 wt. % of final composite coating (about 62 wt. % PMC of final coating). When this composite coating is spray cast on paperboard, it can achieve a contact angle of about 146±3° (nearly superhydrophobic), and a contact angle hysteresis of about 21±5°. A lower value of hysteresis can be expected for more hydrophobic nano-structured particles, but aqueous dispersions based on hydrophobic fillers are extremely difficult to realize. Other nano-structured particles include fumed silica, hydrophobic titania, zinc oxide, nanoclay, exfoliated graphite nanoplatelet, carbon nano-fibers, and mixtures thereof. Other fillers can include milled glass, calcium carbonate, aluminum trihydrate, talc, antimony trioxide, fly ash, clays, and mixtures of thereof.

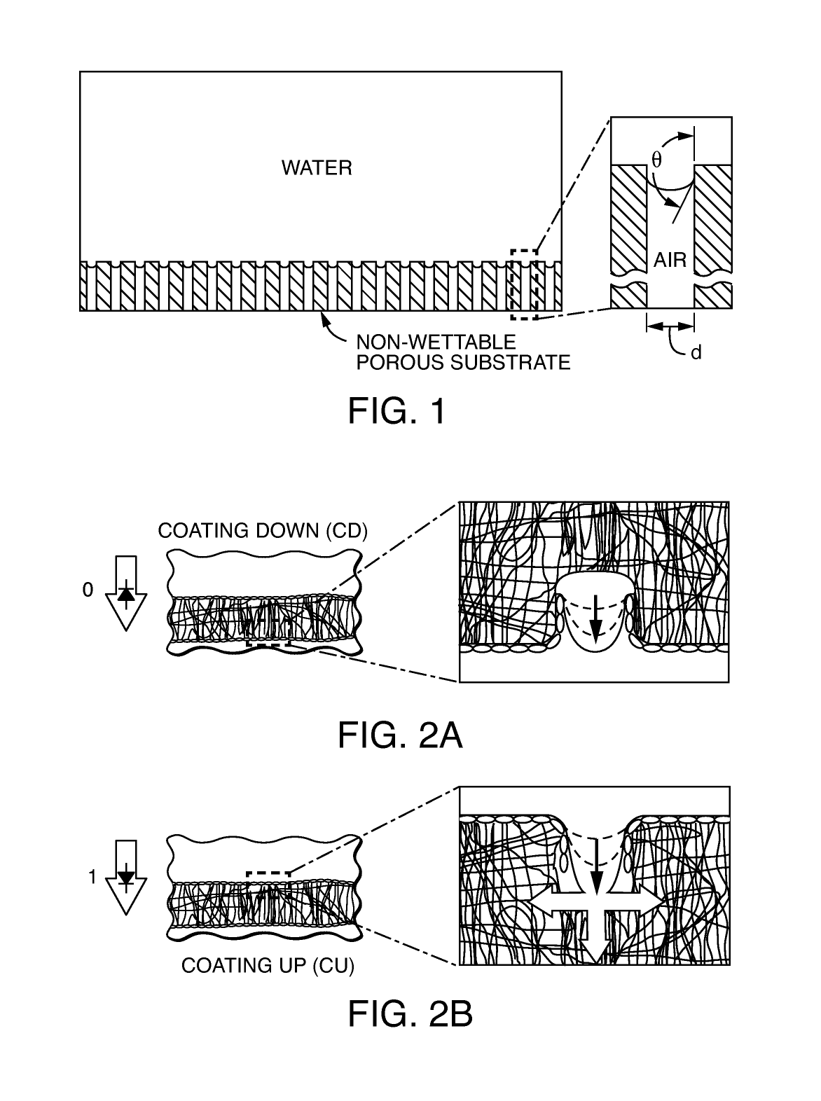

While in the case of superhydrophobicity the emphasis is placed on increasing roughness and lowering surface energy, for resisting penetration of liquids into substrates, substrate pore size and surface energy are important factors. FIG. 1 shows an ideally configured porous substrate (straight pores of uniform diameter d distributed evenly) resisting penetration of water. In this configuration, the pressure necessary for penetration of a hydrophobic substrate with pore size d is given by the Young-Laplace equation Δp=4γ cos θ/d, where g is the surface tension of water, and q (q>90°) is the contact angle between water and the substrate. The more hydrophobic the porous substrate (i.e., the higher the value of q), the higher the liquid penetration pressure Δp. It is apparent that penetration pressure scales inversely with the pore size (the finer the pore, the higher the pressure required to cause water penetration). While pore size can be affected by applying relatively thick coating treatments (other hydrophobic formulations) to porous substrates, the effective pore size after coating is generally predetermined by the pore size of the substrate prior to the coating treatment. The general purpose of applying the coating treatment is to decrease the surface energy of the substrate. In the case of a hydrophilic, cellulosic-based substrate, the coating treatment might not produce a uniform, low-surface energy film around some fibers that, being hydrophilic, can absorb water readily to result in a 0 cm liquid penetration pressure value. Adding coating treatments should confer some appreciable resistance to water penetration. The effectiveness of this approach is measured by the liquid penetration pressure (i.e. “hydrohead”, which is measured in cm of the liquid used to challenge a surface). The higher this pressure is, the more effective the coating method is in imparting hydrophobicity to the substrate. Naturally, the liquid penetration pressure depends on the liquid used (value of γ in the Young-Laplace equation). Because alcohols have lower surface tension than water, mixtures of water and alcohol result in lower penetration pressures.

FIGS. 2A and 2B illustrate the working mechanism of the one-way valve. With the coating up (CU) orientation, meaning the uncoated side is down, droplets applied to the top (the coated side) will sag through pores in the nonwoven until the droplets contact uncoated fibers that wick away the droplet. Conversely, with the coated side down and the uncoated side up (not shown), droplets applied to the top (the uncoated side) will sag into the open space above the coating, but must overcome the full Laplace pressure threshold to completely penetrate through the coated side. The system is analogous to an electronic diode, wherein the transfer of fluid is permitted from one direction but opposed in reverse. As the liquid interface sags into the hydrophobic coated pore from the CU direction, the uncoated hydrophilic fibers beneath will wick the liquid through and allow for fluid transport into the substrate. With the coating down (CD) orientation, the liquid interface can sag under much greater pressures before fluid penetration as there are no uncoated hydrophilic fibers to wick fluid through.

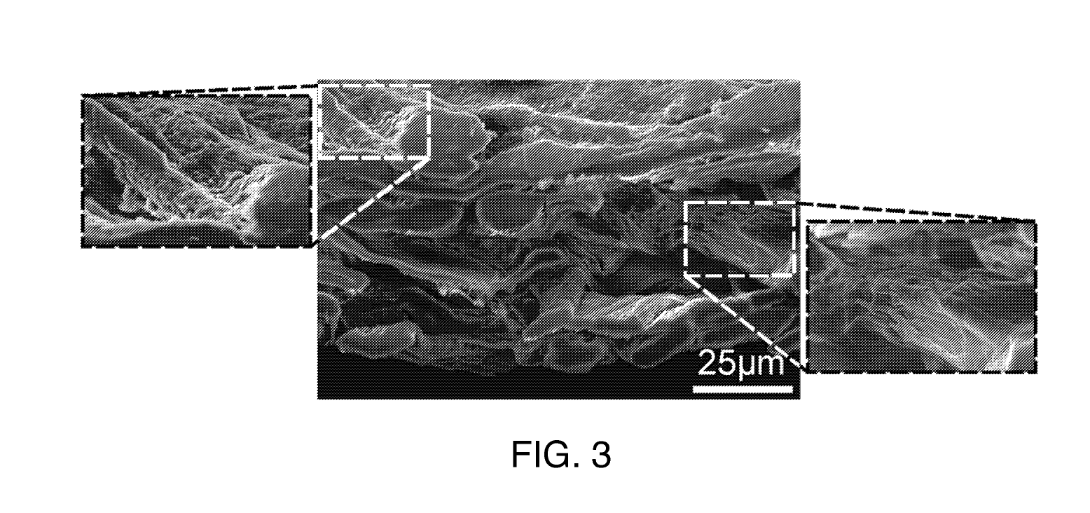

FIG. 3 illustrates a scanning electron microscope (SEM) image of the cross section of a standard paper towel treated as described above, where FIG. 3 is largely analogous to the CU case in schematic diagram of FIG. 2B. The coating is visible at the top of the cross section, along with the gap between the coated and uncoated fibers.

FIGS. 4A and 4B illustrate the phenomenon that substrates introduced to pressures with the coating side up pass fluid more easily than with the coating side down, creating a “valve window.” Samples were tested at increasing coating intervals; the testing shows the resistance difference shrinking at greater gsm coatings. As a result, coating levels should be kept low. It is possible to widen the “valve window” in FIGS. 4A and 4B by refining the precision in spraying, and by creating data points for 0.25 and 0.75 gsm.

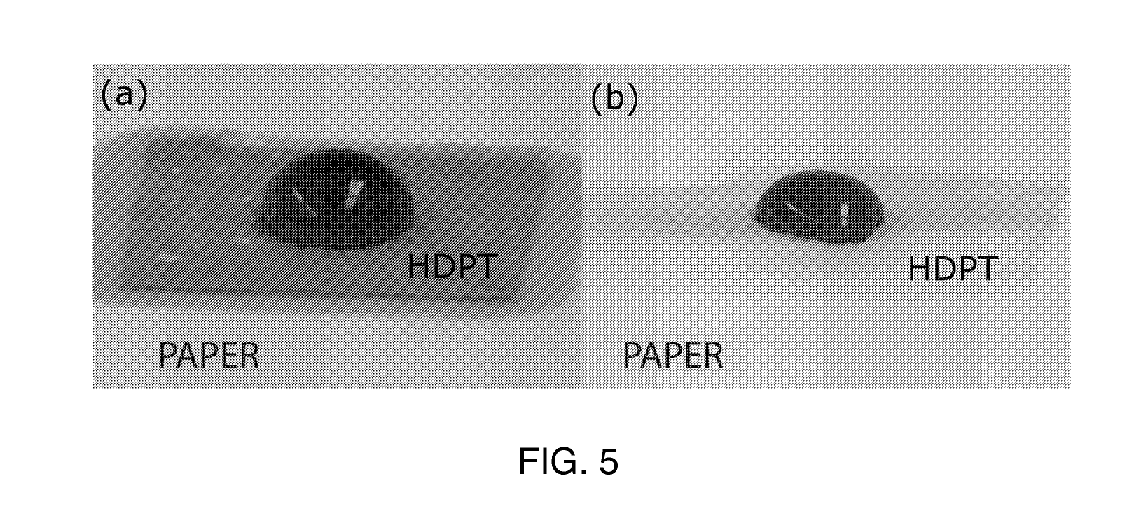

FIG. 5 illustrates the phenomenon of oil-water separation for coated-on-one-side substrates introduced to an oil-water mixture or emulsion. The oil has been dyed red with Oil Red O, and the water has been dyed blue with blue food coloring. The (a) aspect of FIG. 5 illustrates the absorption of the lower surface energy fluid, oil, into the material from the coated side while the relatively higher surface energy fluid, water, has been separated out and rests above the material on the superhydrophobic coating. The (b) aspect of FIG. 5 illustrates the reverse, wherein the uncoated side of the material has been saturated with undyed water. When the same oil-water mixture is introduced to the coated side of the material, water is absorbed and the oil is separated and remains on the surface.

The substrate can be used in an application where it will allow for the separation of oil, or a relatively lower surface energy fluid, from a fluid mixture including a higher surface energy fluid such as water in an oil-water emulsion. Upon contact with an oil-water mixture, water will be repelled by the coating but oil can be absorbed due to its lower surface energy, as shown in the (a) aspect of FIG. 5.

This fluid separation can also be reversed for applications where the lower surface energy fluid is desired from the separation. If the material is introduced to the higher surface energy fluid from the uncoated side thereby saturating the material, such as would be the case if the uncoated side is wetted with water as in the (b) aspect of FIG. 5. The material separates and absorbs the higher surface energy fluid (water in this example) from the mixture, and leaves the lower surface energy fluid (oil in this example) resting on the coated surface, as illustrated in the (b) aspect of FIG. 5.

The present disclosure presents superhydrophobic-coated nonwovens to aid in reducing the presence of body fluids on the body-facing surface of the topsheet, making it more likely for the body fluid to gravitate towards the absorbent core.

### EXAMPLES

The following examples further describe and demonstrate aspects within the scope of the present disclosure. The examples are given solely for the purpose of illustration and are not to be construed as limitations of the present disclosure, as many variations thereof are possible without departing from the spirit and scope of the disclosure.

Specifically, the examples describe using high-density paper toweling (HDPT) available from Kimberly-Clark Professional of Roswell, Ga., SCOTT brand paper toweling (SPT), and BCW surge materials as the substrates, while the superhydrophobic chemistry used is that described in co-pending U.S. patent applications Ser. Nos. 13/193,065 and 13/193,145. The specific HDPT used is KLEENEX brand hard roll towel 50606 with 22.3 lb/2880 square feet or 38 gsm. SPT is an UCTAD tissue with a density lower than that of HDPT. At these low levels of add-on on one surface of the substrate, one-way valve characteristics are demonstrated. The treated side demonstrates a beading followed by penetration through the pores between the treated fibers into the other side of the substrate, while the untreated side demonstrates absorption and spreading of the liquid along the untreated side, but the liquid does not penetrate back through the treated side.

A superhydrophobic formulation was sprayed on a substrate surface to create one-way valve performance. Previous efforts in coating substrates used high coating levels such that the coated surface acted as a barrier to prevent fluid from pass through. At such high coating levels, both fibers and pores between the fibers on the treated surface are covered by the superhydrophobic coating chemicals that actually form a continuous water impermeable film on the substrate. It was discovered that reducing the coating weight to a very low level, such as 2 gsm or lower, caused the coated substrate to exhibit one-way valve properties. The HDPT, SPT, and BCW surge material were coated with a superhydrophobic formulation (1.25% PMC, 1.25% nanoclay and 97.5% water) at a basis weight of 1 gsm and exhibiting one-way valve behavior; water can easily penetrate from the coated side into the uncoated side but cannot penetrate in the opposite direction except under high pressure.

FIG. 3 demonstrates that the coating is only covering fibers on the surface layer and there is no coating chemical to block and bridge over pores between fibers. This coating structure is critical in enabling one-way valve behavior. Although not limited to any particular theory, it is surmised that when the coated side is up, water droplets will be formed on the coated surface, driven by repellency from the coated fibers. These droplets will initially remain above the open pores, and then will sag into the pores until they contact an uncoated fiber below the coated surface. Wicking action by the uncoated fibers will draw the droplets through the pores and away from the coated side. In this manner, the fiber network in the uncoated side provides continuous driving force to remove fluid from the intake point. Conversely, when fluid is added to the uncoated side (i.e. when the coated side is down in this example), a fluid droplet immediately wicks into the fiber network along the uncoated layer. When the droplets finally contact the coated fibers, they must overcome the full Laplace pressure threshold to be able to penetrate through the coated layer because there are no uncoated fibers on the other side of the pores to draw the fluid through.

As described above, FIGS. 4A and 4B reflect the effect of coating level on hydrohead pressure for HDPT and SPT, respectively. The coated-on-one-side substrate introduced to pressures with the coating side up passes fluid more easily than with the coating side down; this creates the valve window. The valve window determines one-way valve performance. The larger the window, the more effective the one-way valve performance exhibited by the substrate. The valve window is reduced as coating levels increase. This explains why one-way valve performance is only observed at a reasonably low coating weight; at higher coating weights the valve window disappears.

In a first specific aspect, a material having one-way valve properties includes a nonwoven substrate having a first surface having a first surface hydrohead value and a second surface having a second surface hydrohead value, and a superhydrophobic formulation disposed on the first surface, wherein the first surface hydrohead value is less than about 1 cm, and wherein the second surface hydrohead value is at least 4 cm greater than the first surface hydrohead value.

A second specific aspect includes the first specific aspect, wherein the superhydrophobic formulation is present at an add-on level of less than about 2 gsm.

A third specific aspect includes the first or second specific aspects, wherein the superhydrophobic formulation includes a hydrophobic component, nano-structured particles, and water.

A fourth specific aspect includes any of the preceding specific aspects, wherein the hydrophobic component is selected from the group consisting of fluorinated polymers, perfluorinated polymers, non-fluorinated polymers, and mixtures thereof.

A fifth specific aspect includes any of the preceding specific aspects, wherein the nano-structured particles are selected from the group consisting of fumed silica, hydrophobic titania, zinc oxide, nanoclay, exfoliated graphite nanoplatelet, carbon nano-fibers, and mixtures thereof.

A sixth specific aspect includes any of the preceding specific aspects, wherein the hydrophobic component is a water-dispersible hydrophobic polymer.

A seventh specific aspect includes any of the preceding specific aspects, wherein the water-dispersible hydrophobic polymer comprises a comonomer selected from acrylic monomers, acrylic precursors, and the like.

An eighth specific aspect includes any of the preceding specific aspects, wherein the superhydrophobic formulation is a modified perfluorinated polymer.

A ninth specific aspect includes any of the preceding specific aspects, the superhydrophobic formulation further comprising a surfactant selected from nonionic, cationic, and anionic surfactants.

A tenth specific aspect includes any of the preceding specific aspects, the superhydrophobic formulation further comprising a stabilizing agent selected from the group consisting of long chain fatty acids, long chain fatty acid salts, ethylene-acrylic acid, ethylene-methacrylic acid copolymers, sulfonic acid, acetic acid, and the like.

An eleventh specific aspect includes any of the preceding specific aspects, the superhydrophobic formulation further comprising a filler selected from the group consisting of milled glass, calcium carbonate, aluminum trihydrate, talc, antimony trioxide, fly ash, clays, and mixtures thereof.

A twelfth specific aspect includes any of the preceding specific aspects, wherein the nonwoven substrate is absorbent.

A thirteenth specific aspect includes any of the preceding specific aspects, wherein the nonwoven substrate is selected from paper toweling, spunbond, meltblown, coform, air-laid, bonded-carded web materials, hydroentangled (spunlace) materials, combinations thereof, and the like.

A fourteenth specific aspect includes any of the preceding specific aspects, wherein the second surface hydrohead value is less than about 16 cm greater than the first surface hydrohead value

A fifteenth specific aspect includes any of the preceding specific aspects, wherein the material is configured to separate a mixture of fluids having differing surface energies on the first surface by absorbing the lower surface energy fluid while leaving the higher surface energy fluid on the first surface of the material.

A sixteenth specific aspect includes any of the preceding specific aspects, wherein the material is configured to separate a mixture of fluids having differing surface energies on the first surface by absorbing the higher surface energy fluid while leaving the lower surface energy fluid on the first surface of the material after wetting the second uncoated surface with the higher surface energy fluid.

In a seventeenth specific aspect, a material having one-way valve properties includes a nonwoven substrate having a first surface having a first surface hydrohead value and a second surface having a second surface hydrohead value, and a superhydrophobic formulation disposed on the first surface at an add-on level of less than about 2 gsm, wherein the second surface hydrohead value is at least 4 cm greater than the first surface hydrohead value.

An eighteenth specific aspect includes the seventeenth specific aspect, wherein the superhydrophobic formulation includes a hydrophobic component, nano-structured particles, and water.

A nineteenth specific aspect includes any of the preceding specific aspects, wherein the hydrophobic component is selected from the group consisting of fluorinated polymers, perfluorinated polymers, non-fluorinated polymers, and mixtures thereof.

In a twentieth specific aspect, a personal care article includes a nonwoven fluid permeable topsheet having a body-facing surface and an opposing backside surface, a fluid impermeable backsheet and at least one intermediate layer disposed therebetween, wherein fluid permeable topsheet includes a nonwoven substrate having a first surface having a first surface hydrohead value and a second surface having a second surface hydrohead value; and a superhydrophobic formulation disposed on the first surface, wherein the first surface hydrohead value is less than about 1 cm, and wherein the second surface hydrohead value is at least 4 cm greater than the first surface hydrohead value.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Disclosure are, in relevant part, incorporated herein by reference; the citation of any document is not to be construed as an admission that it is prior art with respect to the present disclosure. To the extent that any meaning or definition of a term in this written document conflicts with any meaning or definition of the term in a document incorporated by reference, the meaning or definition assigned to the term in this written document shall govern.

While particular aspects of the present disclosure have been illustrated and described, it would be obvious to those skilled in the art that various other changes and modifications can be made without departing from the spirit and scope of the disclosure. It is therefore intended to cover in the appended claims all such changes and modifications that are within the scope of this disclosure.

What is claimed is:

1. A material having one-way valve properties, the material comprising:
a nonwoven substrate having a first surface having a first surface hydrohead value and a second surface having a second surface hydrohead value; and
a superhydrophobic formulation disposed on the first surface, wherein the first surface hydrohead value is less than about 1 cm, and wherein the second surface hydrohead value is at least 4 cm greater than the first surface hydrohead value.

2. The material of claim 1, wherein the superhydrophobic formulation is present at an add-on level of less than about 2 gsm.

3. The material of claim 1, wherein the superhydrophobic formulation includes a hydrophobic component, nano-structured particles, and water.

4. The material of claim 3, wherein the hydrophobic component is selected from the group consisting of fluorinated polymers, perfluorinated polymers, non-fluorinated polymers, and mixtures thereof.

5. The material of claim 4, wherein the nano-structured particles are selected from the group consisting of fumed silica, hydrophobic titania, zinc oxide, nanoclay, exfoliated graphite nanoplatelet, carbon nano-fibers, and mixtures thereof.

6. The material of claim 3, wherein the hydrophobic component is a water-dispersible hydrophobic polymer.

7. The material of claim 6, wherein the water-dispersible hydrophobic polymer comprises a comonomer selected from acrylic monomers, acrylic precursors, and the like.

8. The material of claim 1, wherein the superhydrophobic formulation is a modified perfluorinated polymer.

9. The material of claim 1, the superhydrophobic formulation further comprising a surfactant selected from nonionic, cationic, and anionic surfactants.

10. The material of claim 1, the superhydrophobic formulation further comprising a stabilizing agent selected from the group consisting of long chain fatty acids, long chain fatty acid salts, ethylene-acrylic acid, ethylene-methacrylic acid copolymers, sulfonic acid, acetic acid, and the like.

11. The material of claim 1, the superhydrophobic formulation further comprising a filler selected from the group consisting of milled glass, calcium carbonate, aluminum trihydrate, talc, antimony trioxide, fly ash, clays, and mixtures thereof.

12. The material of claim 1, wherein the nonwoven substrate is absorbent.

13. The material of claim 1, wherein the nonwoven substrate is selected from paper toweling, spunbond, meltblown, coform, air-laid, bonded-carded web materials, hydroentangled (spunlace) materials, combinations thereof, and the like.

14. The material of claim 1, wherein the second surface hydrohead value is less than about 16 cm greater than the first surface hydrohead value

15. The material of claim 1, wherein the material is configured to separate a mixture of fluids having differing surface energies on the first surface by absorbing the lower surface energy fluid while leaving the higher surface energy fluid on the first surface of the material.

16. The material of claim 15, wherein the material is configured to separate a mixture of fluids having differing surface energies on the first surface by absorbing the higher surface energy fluid while leaving the lower surface energy fluid on the first surface of the material after wetting the second uncoated surface with the higher surface energy fluid.

17. A material having one-way valve properties, the material comprising:
a nonwoven substrate having a first surface having a first surface hydrohead value and a second surface having a second surface hydrohead value;
a superhydrophobic formulation disposed on the first surface at an add-on level of less than about 2 gsm, wherein the second surface hydrohead value is at least 4 cm greater than the first surface hydrohead value.

18. The material of claim 17, wherein the superhydrophobic formulation includes a hydrophobic component, nano-structured particles, and water.

19. The material of claim 18, wherein the hydrophobic component is selected from the group consisting of fluorinated polymers, perfluorinated polymers, non-fluorinated polymers, and mixtures thereof.

20. A personal care article comprising a nonwoven fluid permeable topsheet having a body-facing surface and an opposing backside surface, a fluid impermeable backsheet and at least one intermediate layer disposed therebetween, wherein fluid permeable topsheet comprises a nonwoven substrate having a first surface having a first surface hydrohead value and a second surface having a second surface hydrohead value; and
a superhydrophobic formulation disposed on the first surface, wherein the first surface hydrohead value is less than about 1 cm, and wherein the second surface hydrohead value is at least 4 cm greater than the first surface hydrohead value.

21. The personal care article of claim 20, wherein the nonwoven fluid permeable topsheet is selected from paper toweling, spunbond, meltblown, coform, air-laid, bonded-carded web materials, hydroentangled (spunlace) materials, combinations thereof, and the like.

22. The personal care article of claim 20, wherein the superhydrophobic formulation is a modified perfluorinated polymer.
